(19) United States
(12) Patent Application Publication
(10) Pub. No.: US 2011/0082361 A1
(43) Pub. Date: Apr. 7, 2011

(54) METHOD FOR CONTROLLING AN IMAGING EXAMINATION SYSTEM

(76) Inventors: Kirstin Jattke, Nurnberg (DE); Diana Martin, Herzogenaurach (DE)

(21) Appl. No.: 12/896,152

(22) Filed: Oct. 1, 2010

(30) Foreign Application Priority Data
Oct. 2, 2009 (DE) 10 2009 048 151.6

(51) Int. Cl. A61B 5/055; G01N 23/02

(52) U.S. Cl. 600/410; 250/491.1; 378/208

(57) ABSTRACT

A method is disclosed for controlling an imaging examination system for examination of a patient. In at least one embodiment, the imaging examination system includes a first imaging examination device with a first isocenter and a second imaging examination device with a second isocenter. The examination system further includes an examination table which can be positioned in respect of the isocenters. In at least one embodiment of the method, an examination region of the patient is determined and at least one examination table position and a first field of view in respect of the first isocenter and a second field of view in respect of the second isocenter is automatically determined as a function of the examination region and the isocenters such that, in the examination table position, a capture of a first item of image information in the first field of view with the first imaging examination device and a capture of a second item of image information in the second field of view with the second imaging examination device is possible.

### PRIORITY STATEMENT

The present application hereby claims priority under 35 U.S.C. §119 on German patent application number DE 10 2009 048 151.6 filed Oct. 2, 2009, the entire contents of which are hereby incorporated herein by reference.

### FIELD

At least one embodiment of the present invention generally relates to a method for controlling an imaging examination system for examination of a patient, in particular a method for controlling so-called hybrid systems which permit an examination using two or more different examination methods.

### BACKGROUND

In medical imaging so-called hybrid systems are increasingly frequently used, such as for example PET/CT (Positron Emission Tomography/Computed Tomography), SPECT/CT (Single Photon Emission Computed Tomography/Computed Tomography), MR/PET (Magnetic Resonance Tomography/Positron Emission Tomography) and MR/SPECT (Magnetic Resonance Tomography/Single Photon Emission Computed Tomography). In such hybrid systems two examination devices can for example be disposed in one system. Advantageously for example an examination device with a high spatial resolution, for example MR or CT, is combined with an examination device with a high sensitivity, for example a nuclear medicine examination device, for example SPECT or PET.

The various examination devices combined with one another normally have different fields of view (FoV) in the Z direction, in other words in the direction of movement of an examination table on which a patient lies. Because of these different fields of view examination settings, in particular for multi-step examinations in which the patient is examined in various positions along the Z direction, are difficult and complex for a user of the hybrid system if the best possible anatomical coverage, for example of a region of the body, of the whole body or of an individual organ, for example the liver, is to be achieved, along with examination times which are as short as possible. In PET/CT examinations the individual examinations are therefore for example performed consecutively.

### SUMMARY

In at least one embodiment of the invention, an improved method is provided for controlling an imaging hybrid examination system.

According to at least one embodiment of the present invention, a method for controlling an imaging examination system is disclosed and by an imaging examination system is disclosed. Advantageous and preferred embodiments are described in the dependent claims.

According to at least one embodiment of the invention a method for controlling an imaging examination system is provided for examination of a patient. The imaging examination system comprises a first imaging examination device with a first isocenter and a second imaging examination device with a second isocenter and an examination table on which the patient lies and which can be positioned in respect of the isocenters. Such examination systems are also referred to as hybrid systems. The first and second imaging examination device can for example be designed to perform nuclear spin tomography, x-ray tomography, positron emission tomography or single photon emission computed tomography. The examination table on which the patient lies can be positioned such that it passes through various positions relative to the isocenters of the imaging examination devices. In the method an examination region of the patient is determined and as a function of this examination region and the isocenters of the examination devices one or more examination table positions are automatically determined. For each examination table position a first field of view (FoV) is automatically determined, in relation to the first isocenter, as is a second field of view in relation to the second isocenter, as a function of the examination region and the isocenters. The examination table position and the associated fields of view are determined such that in the examination table position it is possible to capture a first item of image information in the first field of view using the first imaging examination device and to capture a second item of image information in the second field of view using the second imaging examination device.

Because examination table positions and associated first and second fields of view are automatically determined, items of image information of the first and the second imaging examination device can be captured simultaneously in each of the determined examination table positions, as a result of which an examination time can be reduced. Thanks to the automatic determination a user of the hybrid examination system has less to do and a planning phase for an examination is shortened.

According to one embodiment the examination region is automatically determined in an already captured image of the patient on the basis of a user input of a start point and an end point in a direction of movement of the examination table. The examination table position and the associated first and second fields of view can be determined such that the examination region is fully covered by a totality of the first fields of view and/or a totality of the second fields of view. As a result, handling a hybrid examination system can be considerably simplified. After the start point and the end point have been input with the aid for example of a graphical user interface, the examination region is automatically defined between the start and the end point, and examination table positions suitable for this examination region, and associated fields of view, in other words recording regions, of the first and second imaging examination devices are determined.

Alternatively the examination region can also be automatically determined on the basis of a user setting of markers on the patient or on the examination table. In yet another embodiment the examination region can be automatically determined on the basis of a user selection of an organ of the patient in an already captured and automatically segmented image of the patient.

The determined examination region and the first and second automatically determined fields of view can be displayed on a display of the examination system for a user of the examination system. As a result the user of the examination system obtains an overview of the totals of the planned examination steps in the individual examination table positions with the associated fields of view of both the examination devices. In addition coverage of the examination region by the first and second fields of view and for example resultant overlapping regions are displayed to the user.

According to a further embodiment the method further comprises automatic positioning of the examination table to the determined examination table positions plus automatic capture of the first item of image information in the first field of view with the first imaging examination device and automatic capture of the second item of image information in the second field of view with the second imaging examination device. In this way, following a user input for example, which results in the determination of the examination region, the automatically determined examination sequence can initially be examined on the display of the examination system and then be automatically performed across several examination table positions. As a result the entire examination sequence can be expedited, as a result of which examination results are available faster, the patient has to spend less time in the examination system and thus an efficient use of the examination system is ensured.

In a further embodiment the examination table position or the multiple examination table positions and the respective first and second fields of view is/are determined such that the respective first field of view and the respective second field of view can be captured simultaneously. The respective first and second field of view can in this case for example capture an identical region of the patient, two different regions of the patient which however overlap, or two different regions which do not overlap. Because both fields of view can be captured simultaneously, an examination time can be reduced.

According to a further embodiment a user of the examination system can, by means of a user input, define a subregion in the examination region, which can be captured with one or both examination devices without repositioning the examination table. On the basis of this user input an examination table position and a corresponding first field of view for the first imaging examination device and a second field of view for the second imaging examination device are automatically determined such that the entire subregion is covered from the first field of view and/or the second field of view without repositioning the examination table. In certain examinations, for example the examination of an organ, it can be advantageous if a subregion which includes this organ is captured as fast as possible, in other words in particular without repositioning the examination table, since for example the patient should not move while this organ is being captured and for example holds his/her breath for the examination time. Such basic conditions can be taken into account by defining such a subregion during the automatic determination of the examination table positions and of the corresponding fields of view. This can then also be used particularly advantageously if the subregion is to be captured simultaneously by both examination devices.

According to a further embodiment the examination table positions and the respective first and second fields of view can be determined such that an examination period for the determined examination region is minimized. To this end large fields of view can for example be selected in the respective isocenters. Alternatively the examination table positions and the respective first and second fields of view can be determined such that an image quality of the first and/or second item of image information is maximized. In this case more table positions can be determined, compared for example with the table positions optimized to the examination period, in order to arrange the fields of view in optimum regions of the isocenters and thus achieve as good an image quality as possible.

According to an embodiment of the present invention an imaging examination system is furthermore provided for examination of a patient. The imaging examination system comprises a first imaging examination device with a first isocenter, a second imaging examination device with a second isocenter, an examination table on which the patient lies and which can be positioned in respect of the isocenters and passes through various positions in respect of the isocenters, a user interface for the input and output of information by or for a user, and a control device. The control device determines on the basis of a user input an examination region of the patient and determines as a function of the examination region and the isocenters one or more examination table positions and for each examination table position a first field of view in relation to the first isocenter and a second field of view in relation to the second isocenter. The examination table positions and the corresponding first and second fields of view are determined such that in a respective examination table position it is possible to capture a first item of image information in the first field of view with the first imaging examination device and to capture a second item of image information in the second field of view with the second imaging examination device.

The imaging examination system can further be designed such that it is suitable for performing the previously described method and embodiments thereof, and hence also comprises the previously described advantages.

### BRIEF DESCRIPTION OF THE DRAWINGS

Embodiments of the present invention is described in detail below with reference to the enclosed drawings.

FIG. 1 shows in diagrammatic form an imaging examination system according to one embodiment of the present invention.

FIG. 2 shows in diagrammatic form different isocenters in a Z direction and fields of view from two different imaging examination devices for various table positions.

FIG. 3 shows automatically determined fields of view for various table positions.

FIG. 4 shows a input of seed points for an automatic determination of fields of view.

FIG. 5 shows an automatically determined examination region.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

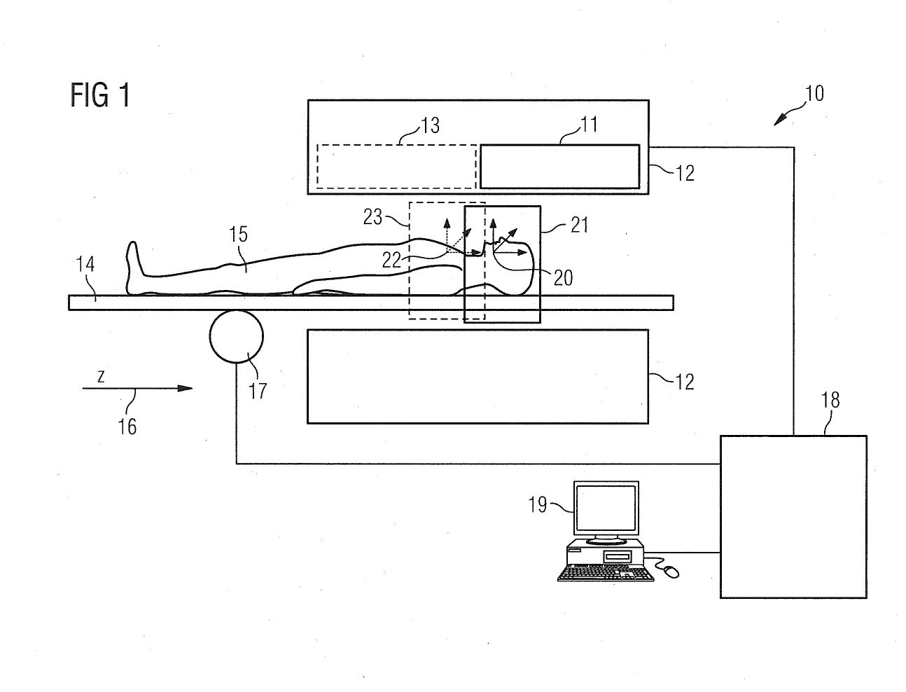

FIG. 1 shows a hybrid examination system 10 with two imaging examination devices 11, 13. The first imaging examination device 11 can for example be a magnetic resonance tomograph, which further comprises a tube-shaped magnet 12 for generation of its BO field, said magnet being shown in FIG. 1 in a sectional drawing. The second imaging examination device 13 can for example be a positron emission tomograph (PET) or a single photon emission computed tomograph (SPECT). The second examination device 13 is likewise disposed in the tube-shaped magnet 12. The arrangement of the examination devices 11, 13 in FIG. 1 is only shown diagrammatically. The components of the first and second examination devices 11, 13 can be nested in one another in any order and can be disposed so as to overlay one another or to be completely separate. The examination system 10 further comprises an examination table 14 on which a patient 15 can be disposed. The examination table 14 can be positioned in relation to the examination devices 11, 13 in an inner cavity of the magnet 12 in the Z direction, which is indicated by the arrow 16. The positioning of the examination table 12 can be performed with the aid of a drive 17, for example an electric drive, by a control device 18 which is linked to the drive 17. The controller 18 of the examination system 10 is additionally linked to the examination devices 11, 13 and a user interface 19. The user interface 19 can for example be a computer with a graphical interface, a keyboard and a pointer input tool, a mouse for example.

The first examination device 11 has a first isocenter 20, which in the case of a magnetic resonance tomograph for example corresponds to the midpoint of the cylindrical borehole in the magnet 12. In respect of the isocenter 20 it is possible, if the examination device 11 is suitably controlled, to set a recording volume range 21, also referred to as a field of view (FoV). With a magnetic resonance tomograph it is possible to define a size of the field of view 21 in order to set a resolution within the field of view 21 and thus an image quality. In addition the size of the field of view 21 can also be used to change a recording speed which is required in order to record image data in the field of view 21.

The second imaging examination device 13 has an isocenter 22 which can coincide with the isocenter 20 of the first examination device 11 or can be at a distance from it. In the case of a positron emission tomograph the isocenter can for example be a midpoint of a cylindrical space inside a tube-shaped detector system of the positron emission tomograph. A second field of view 23 of the positron emission tomograph 13 is defined in respect of the isocenter 22. The field of view 23 is essentially predefined by the extension of the detector system in the Z direction, although in regions which are further from the isocenter 22 in the Z direction an image quality of the second examination device can decrease, since particularly in the case of positron emission tomographs in a 3D recording mode the sensitivity decreases at the edge. Hence also in the case of a positron emission tomograph restricting the field of view 23 in the Z direction is a suitable way of improving the image quality.

To increase the distinction between the two examination devices 11, 13 and their associated isocenters 20, 22 and fields of view 21, 23 in FIG. 1, the examination device 13 as well as the associated isocenter 22 and the associated field of view 22 are drawn with dashed lines.

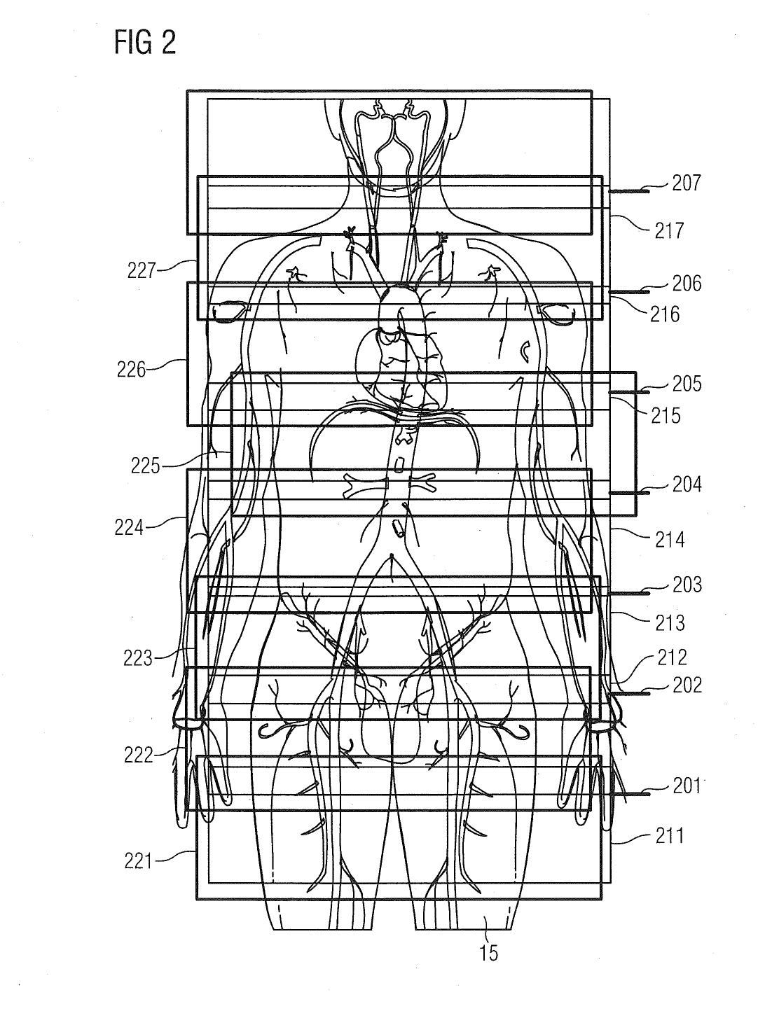

FIG. 2 shows for various table positions of the examination table 14 the position of the isocenter 20 of a first imaging examination device 11, for example a magnetic resonance examination device, and the associated fields of view 21 as well as corresponding fields of view 23 of the second imaging examination device 13. For a first table position the isocenter of the first imaging examination device is located in the Z direction for example at the position shown by the line 201. The associated field of view of the first imaging examination device is shown by the rectangle 211 in FIG. 2 in respect of a recording of the patient 15. For this table position the second imaging examination device has a field of view which is characterized by the rectangle 221 in FIG. 2. For a further table position the isocenter of the first imaging examination device is located in the Z direction at the point characterized with 202 and the corresponding fields of view of the first and second imaging examination device are characterized by the rectangles 212 and 222 respectively. For further table positions the isocenters 203-207 of the first imaging examination device as well as corresponding fields of view 213 to 217 of the first examination device and corresponding fields of view 223-227 of the second examination device are shown.

It is apparent from FIG. 2 that it is a very complex task for a user to coordinate the different fields of view with one another in the Z direction in the case of a multistep examination in order to achieve the best possible anatomical coverage, in particular for individual organs, for example the liver, and simultaneously to ensure as fast as possible an examination, in other words a short examination time. For this reason in general the recordings of such a hybrid system are performed consecutively, so that it is not necessary to adjust the fields of view of the two examination devices 11 and 13.

According to at least one embodiment of the present invention the coordination of the fields of view 21 and 23 of the two examination devices 11 and 13 respectively and the associated determination of the table positions in the Z direction of the examination table 14 are therefore performed automatically by the control device 18.

According to one embodiment the user for example inputs a start point and an end point of a desired examination region. This input of start and end point of the examination region can for example be performed in an already recorded image or a video image of the patient or by setting markers on the patient or the examination table or by defining table positions. The control device 18 then automatically determines a requisite number of examination steps and for each examination step a corresponding table position and the corresponding fields of view of the two examination devices 11 and 13 respectively. In this case corresponding overlapping regions of the fields of view 21 and 23 respectively between the individual examination steps and table positions are taken into account, as are necessary for generating a complete recording of the desired examination region. In addition the number of table positions and an orientation of the individual table positions to one another can be automatically selected by the control device 18 such that simultaneous recording with both examination devices 11, 13 is possible for each station of the examination table. The automatic determination of these table positions takes into account the isocenters 20 and 22 of the examination devices 11 and 13 respectively and their relative position to one another, as they are combined with one another in the hybrid system 10. The automatic determination produces an optimization in respect of image quality and examination time. In this case attributes of the two examination devices 11 and 13 are taken into account, for example minimum and maximum fields of view 21 and 23, restrictions regarding postprocessing by the software of the examination devices 11 and 13, as well as minimum and maximum overlapping regions of the fields of view 21 and 23.

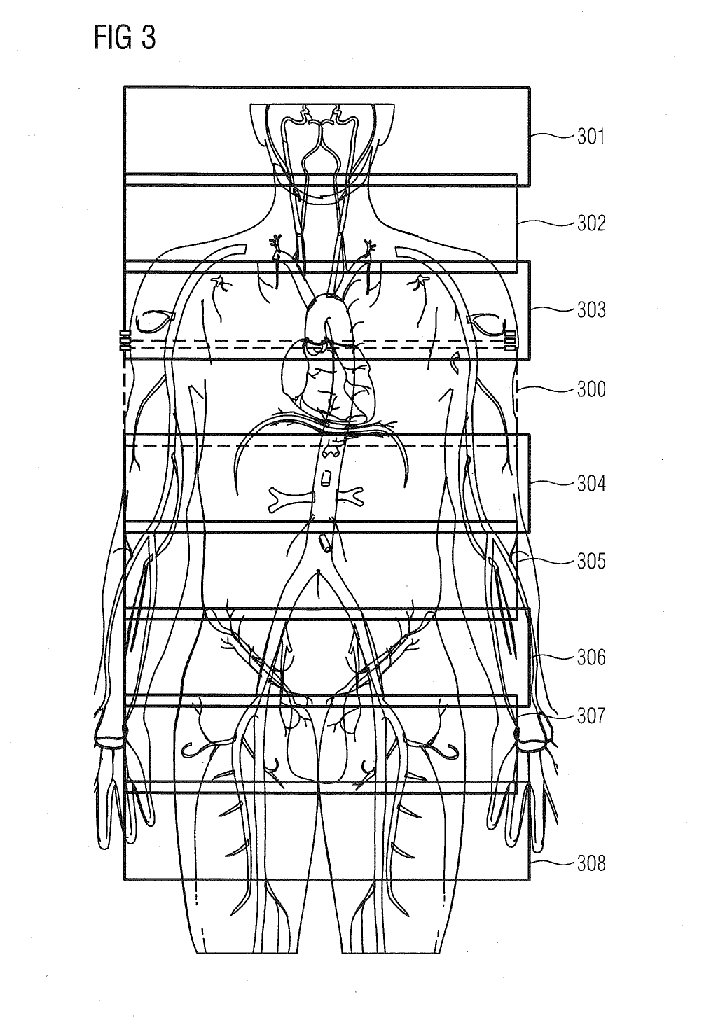

For diagnostic reasons it may be necessary to examine and fully cover a determined organ or several determined organs in an individual step, in other words for one table position. This may for example be necessary on the basis of attributes of the examination device itself, or in order for example to reduce artifacts, which can arise for example due to a movement of the patient, for example when breathing. For this reason the control device 18 takes account, on the basis of an input by the user, of such restrictions. The user can for example directly mark such a region of interest via the user interface 19 in an already recorded image. This is shown for example in FIG. 3 by the region 300 drawn with dashed lines. The user for example defines by marking the region 300 that this region is to be captured in one examination step, in other words in one table position. The control device 18 now determines the further table positions such that the region 300 can be captured in one examination step and regions over and below the region 300 are captured with corresponding overlapping. FIG. 3 therefore shows the regions 301-303 which are captured in sequence, before the region 300 is captured in one step, as well as the regions 304-308 which are captured in sequence after the capture of the region 300.

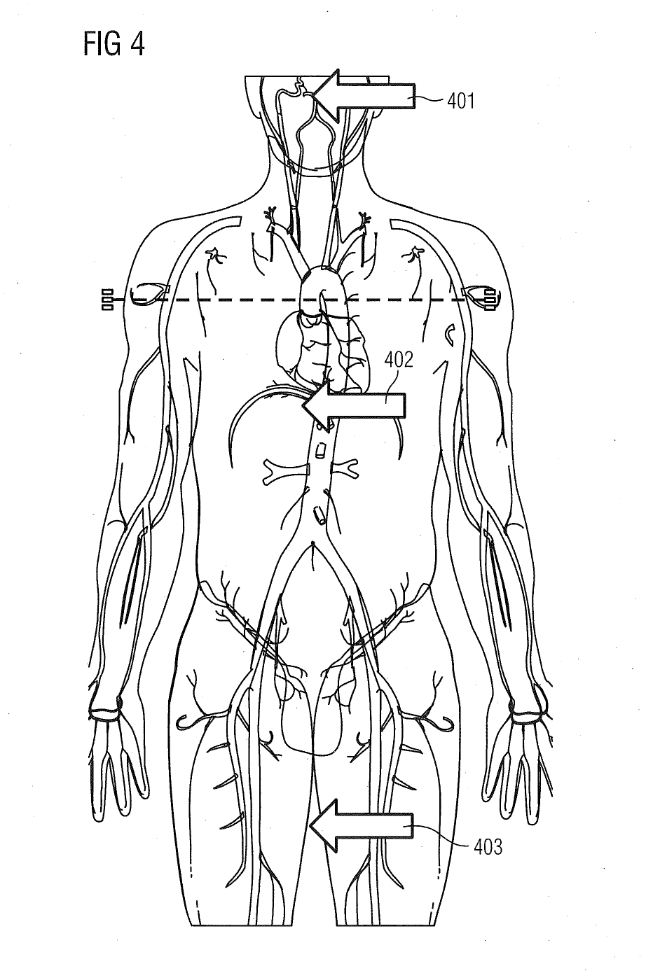

To ensure that an individual organ or a region of interest is captured in one examination step, in other words in one table position, as a so-called SLAB in a three-dimensional imaging operation, the user can also select a determined region or a determined organ by setting a so-called seed point. With the aid of automatic segmentation, which is performed in a previously recorded image of the patient by the control device 18, the user can thus for example select an organ, by selecting a point inside the desired organ with the aid of the user interface 19, and with the aid of segmentation the control device 18 determines the organ region and coordinates table positions and associated fields of view 21 and 23 of the imaging examination devices 11 or 13 respectively with one another such that the desired organ can be recorded in one examination step. FIG. 4 shows by way of example how a user, with the aid of seed point arrows 401-403, can set seed points for example in a magnetic resonance recording of the patient, which then with the aid of automatic segmentation are used to determine the table positions and the fields of view for the multi-step examination.

With the aid of fully automatic segmentation, in which the control device 18 automatically segments organs in for example a magnetic resonance recording and determines their positions, the user can for example select an organ directly which is to be recorded in one examination step. The control device 18 then automatically coordinates the table positions and the associated fields of view 21 and 23 of the examination devices 11 and 13 respectively in a corresponding manner.

The definition of a specific organ can be performed with the aid of recordings of either of the two examination devices 11 and 13. In the case of a hybrid system which for example comprises a magnetic resonance tomograph and a positron emission tomograph, the organ can for example be determined in an image of the positron emission tomograph which shows the main tumor activities, and planning for a magnetic resonance examination can be optimized, in other words the identified region is covered in one step by the magnetic resonance tomograph.

Following the determination of the table positions and the corresponding fields of view 21 and 23 the result can be displayed graphically to a user on a screen of the user interface 19. FIG. 3 shows by way of example such a display, which shows a user the fields of view for the various determined table positions and the corresponding overlapping regions. The user can check prior to the actual performance of the examination whether this division is suitable and if necessary insert corrections, for example by specifying further regions which are to be captured in one examination step. The control device 18 then determines new table positions and corresponding fields of view 21 and 23 and displays them on a screen of the user interface 19. As a result the entire work sequence can be simplified and expedited and it can be simultaneously ensured that user requirements in respect of the examination regions are taken into account.

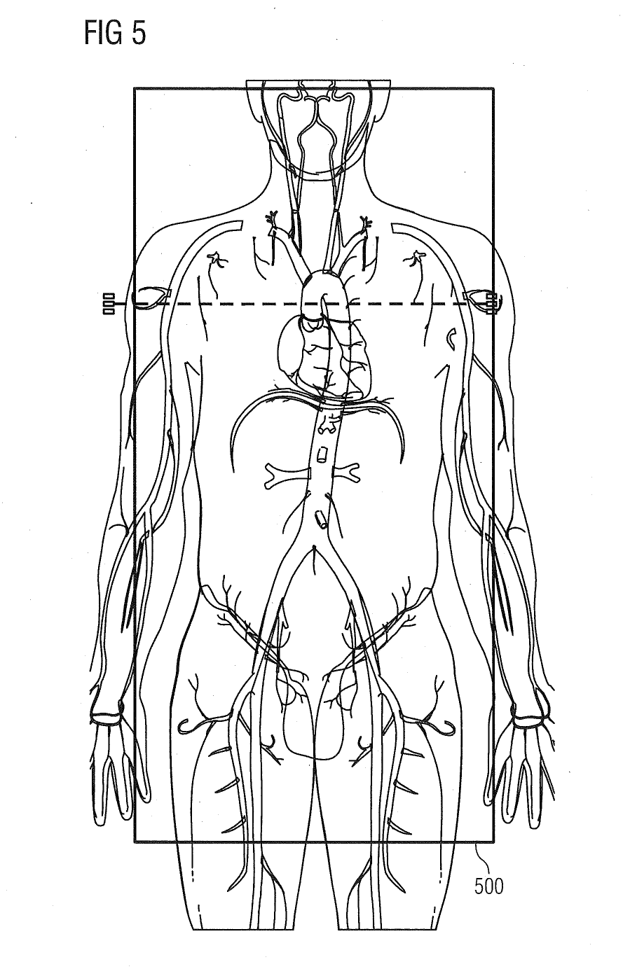

The graphical display for the user on the user interface 19 can additionally for example display the entire examination region, which is covered by the totality of the determined table positions and associated fields of view 21 and 23, by for example a rectangle 500 which delimits the entire examination region, as shown in FIG. 5. In addition for example the markings which the user has input as start and end points for the examination region can be displayed. Furthermore the fields of view of optionally the first examination device 11 or the second examination device 13 or the fields of view of both examination devices 11 and 13 can be displayed jointly for each of the table positions. In addition the fields of view and overlapping regions can be displayed in relation to the determined organs.

With the aid of at least one embodiment of the present invention important aspects of an examination using a hybrid system 10 can thus be improved, for example the image quality or the overall recording time with the aid of simultaneous recording by the two examination devices 11 and 13.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a builtin rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### LIST OF REFERENCE CHARACTERS

10 Examination system
11 First examination device
12 Magnet
13 Second examination device
14 Examination table
15 Patient
16 Z direction
17 Drive
18 Control device
19 User interface
20 First isocenter
21 First field of view
22 Second isocenter
23 Second field of view
201-207 Position of the first isocenter
211-217 First field of view
221-227 Second field of view
300-308 Field of view, examination region
401-403 Seed point arrow
500 Examination region

What is claimed is:

1. A method for controlling an imaging examination system for examination of a patient, the imaging examination system including at least a first imaging examination device with a first isocenter and a second imaging examination device with a second isocenter and including an examination table, positionable in respect of at least one of the isocenters and on which the patient lies, to pass through various positions relative to the imaging examination system, the method comprising:
determining an examination region of the patient; and
automatically determining at least one examination table position, a first field of view in respect of the first isocenter and of a second field of view in respect of the second isocenter as a function of the examination region and the isocenters such that in the at least one examination table position, a capture of a first item of image information in the first field of view with the first imaging examination device and a capture of a second item of image information in the second field of view with the second imaging examination device is possible.

2. The method as claimed in claim 1, wherein the at least one examination table position and the respective first and second fields of view are determined such that at least one of the examination region is fully covered by a totality of the first fields of view, and the examination region is fully covered by a totality of the second fields of view.

3. The method as claimed in claim 1, further comprising:
displaying at least one of the examination region, the first field of view and the second field of view on a display of the examination system.

4. The method as claimed in claim 1, further comprising:
automatically positioning the examination table to the at least one examination table position;
automatically capturing the first item of image information in the first field of view with the first imaging examination device; and
automatically capturing the second item of image information in the second field of view with the second imaging examination device.

5. The method as claimed in claim 1, wherein on the basis of a user input of a start point and an end point in a direction of movement of the examination table in an already captured image of the patient, the examination region is automatically determined.

6. The method as claimed in claim 1, wherein on the basis of a user setting of markers on the patient or on the examination table, the examination region is automatically determined.

7. The method as claimed in claim 1, wherein on the basis of a user input of table positions, the examination region is automatically determined.

8. The method as claimed in claim 1, wherein on the basis of a user selection of an organ of the patient in an already captured and automatically segmented image of the patient, the examination region is automatically determined.

9. The method as claimed in claim 1, wherein the at least one examination table position and the respective first and second fields of view are determined such that the respective first field of view and the respective second field of view are simultaneously capturable.

10. The method as claimed in claim 1, wherein on the basis of a user input which defines a subregion in the examination region, the at least one examination table position and the respective first and second fields of view are automatically determined such that the entire subregion is covered by at least one of the respective first field of view and the respective second field of view, without repositioning the examination table.

11. The method as claimed in claim 1, wherein the at least one examination table position and the respective first and second fields of view are determined such that an examination period for the examination region is minimized.

12. The method as claimed in claim 1, wherein the at least one examination table position and the respective first and second fields of view are determined such that an image quality of at least one of the first and second items of image information is maximized.

13. The method as claimed in claim 1, wherein the first imaging examination device is designed to perform nuclear spin to mography, x-ray tomography, positron emission tomography or single photon emission computed tomography.

14. The method as claimed in claim 1, wherein the second imaging examination device is designed to perform nuclear spin tomography, x-ray tomography, positron emission tomography or single photon emission computed tomography.

15. An imaging examination system for examining a patient, comprising
a first imaging examination device including a first isocenter;
a second imaging examination device including a second isocenter;
an examination table, positionable in respect of the isocenters and on which the patient lies, to pass through various positions in respect of the first and second isocenters;
a user interface for the input and output of information by/for a user; and
a control device, designed on the basis of a user input, to determine an examination region of the patient and to determine at least one examination table position and a first field of view in relation to the first isocenter and a second field of view in relation to the second isocenter as a function of the examination region and the isocenters such that, in the at least one examination table position, a capture of a first item of image information in the first field of view with the first imaging examination device and a capture of a second item of image information in the second field of view with the second imaging examination device is possible.

16. An imaging examination system for examining a patient, comprising
a first imaging examination device including a first isocenter;
a second imaging examination device including a second isocenter;
an examination table, positionable in respect of the isocenters and on which the patient lies, to pass through various positions in respect of the first and second isocenters;
a user interface for the input and output of information by/for a user; and
a control device, designed on the basis of a user input, to determine an examination region of the patient and to determine at least one examination table position and a first field of view in relation to the first isocenter and a second field of view in relation to the second isocenter as a function of the examination region and the isocenters such that, in the at least one examination table position, a capture of a first item of image information in the first field of view with the first imaging examination device and a capture of a second item of image information in the second field of view with the second imaging examination device is possible, wherein the examination system is designed to perform the method as claimed in claim 1.

17. The method as claimed in claim 2, further comprising:
displaying at least one of the examination region, the first field of view and the second field of view on a display of the examination system.

18. The method as claimed in claim 2, further comprising:
automatically positioning the examination table to the at least one examination table position;
automatically capturing the first item of image information in the first field of view with the first imaging examination device; and
automatically capturing the second item of image information in the second field of view with the second imaging examination device.

19. The method as claimed in claim 3, further comprising:
automatically positioning the examination table to the at least one examination table position;
automatically capturing the first item of image information in the first field of view with the first imaging examination device; and
automatically capturing the second item of image information in the second field of view with the second imaging examination device.

20. A computer readable medium including program segments for, when executed on a computer device, causing the computer device to implement the method of claim 1.
